(19) United States
(12) Patent Application Publication
(10) Pub. No.: US 2023/0320652 A1
(43) Pub. Date: Oct. 12, 2023

(54) SYSTEM FOR COMPARING THE PHYSIOLOGICAL PARAMETERS OF A PATIENT

(71) Applicant: SKIN PLASTIC LAB S.R.L., Maglie (LE) (IT)

(72) Inventors: Stefano PERTICAROLI, Maglie (LE) (IT); Giovanni PAPA, Maglie (LE) (IT)

(21) Appl. No.: 17/926,304

(22) PCT Filed: May 3, 2021
(86) PCT No.: PCT/IB2021/053668

§ 371 Date: Nov. 18, 2022

(30) Foreign Application Priority Data
May 21, 2020 (IT) 102020000011881

(51) Int. Cl. A61B 5/00; A61B 5/145; A61B 5/01

(52) U.S. Cl. CPC A61B 5/445; A61B 5/14539; A61B 5/14542; A61B 2562/046; A61B 5/6833; A61B 5/442; A61B 5/4842; A61B 5/015

(57) ABSTRACT

System for comparing the physiological parameters of a patient, including: a primary detection device constituted by printed circuits arranged on a flexible support, adapted to be placed at a first skin portion to be diagnosed; the printed circuits including at least a matrix of temperature sensors and a microcontroller; a secondary detection device constituted by printed circuits arranged on a flexible support, adapted to be placed at a second skin portion, healthy, of a patient in order to detect the same data as the first skin portion; the printed circuits including at least a matrix of temperature sensors and microcontroller; a display device in order to allow the display of the data detected by the primary detection devices.

### FIELD OF THE ART

The present invention operates in the field of electronic devices for aiding the diagnostic, medical and more generally health field.

In particular the system of the present invention is adapted to be used for monitoring the vitality of the skin layers, and in a particularly convenient embodiment, in post-operation phase for verifying that a wound is correctly healing.

### PRIOR ART

The temperature of the skin, intended as member constituted by different tissues and sprayed by blood vessels, is a fundamental indicator in the determination of the vitality of the skin itself.

There is a correlation, defined and proven by numerous scientific articles, between the temperature variations of the skin and pathologies which affect the skin and also the deep circulation in some cases, such as those relative to the limbs. For example, temperature gradients in skin areas are observed in patients with vascular disorders which have an irregular blood flow in the affected zones. The current systems existing for thermographic imaging, capable of detecting temperature gradients and/or of monitoring blood flow variations of tissues and/or organs, are based on infrared thermography or on the perfusion of tracers.

In the case of infrared thermography, the method for transmitting heat between the source, represented by a tissue and/or organ volume, is detected by means of irradiation. This type of transmission and detection of the temperature is intrinsically non-selective since it is capable of determining the temperature of a body not in contact with the sensor, limiting the discrimination capacity at the average temperature of a volume with accuracy levels at most on the order of tenths of centigrade degrees.

In the case of the perfusion of intravenous tracers for the determination of the blood flow variations, indocyanine green or fluorescein are considered. This type of transmission and detection of the information relative to the blood flows is based on radiations in the optical or near-optical spectrum (ultraviolet), by means of auxiliary machinery, such as microscopes or microchambers sensitive to specific wavelengths. The latter two methods, even if they have greater accuracies, are invasive treatments for the patient since the tracers must be perfused and do not give information directly correlated to the temperature. Finally, they do not allow the monitoring of the magnitudes of interest in real time and for a prolonged time period.

In the same field of application as the present patent application, several patents are already present, such as for example the patent U.S. 2001/206655 which describes a bandage that incorporates a matrix of temperature sensors fixable to the body of a patient. The bandage can contain electronic processing components and a transmitter. Another patent WO2009/144615 claims a device for the electrical treatment of a wound which comprises: a flexible substrate, provided with a matrix of electrodes, adapted to be attached to the skin wound; means for determining the presence of the wound and its perimeter; means for controlling a liquid load between the matrix and the wound and means for applying an electrical voltage between the wound and the surrounding skin. Other known documents claim the use of temperature sensors for sanitary and diagnostic purposes. In particular the patent U.S. 2018/183794 describes a method for determining the emergence of an ulcer on a foot of a patient starting from the detections carried out by a plurality of temperature sensors situated on a device configured for receiving at least a foot.

Indeed, patent U.S. 2016/183794 describes a temperature detector bandage which includes: a sealed and flexible battery comprising a printed electrochemical cell, a flexible circuit which includes a microprocessor, a temperature sensor, a wireless transmitter and an antenna for transmitting the detected data to a remote device.

The technical problem raised by the Applicant regards the fact that the detection of the temperature data only in the vicinity of the wound could be altered in the case of an increase or a decrease of the temperature throughout the entire body of the patient. In addition, the limitation of the indications of correct or poor healing to only the temperature data seems to provide an incomplete and easily erroneous diagnosis.

From the study of the prior art, neither documents nor devices have been found which are capable of executing analyses of all the important physiological parameters for ascertaining the correct healing of a wound and the correct blood circulation, both in proximity to a wound and in portions of healthy skin. Nor have there been mentions of devices that can compare the physiological parameters detected in the portion to be diagnosed with the general parameters of the patient, detected from body portions that are far from or extraneous to the wound or to the presumed circulatory problem.

Object of the present invention is, therefore, that of filling this gap of the prior art, by proposing a new and innovative system for comparing the physiological parameters of a patient which overcomes the critical factors encountered and which allows an accurate diagnosis of the state of healing of a wound and of possible circulatory problems.

### DESCRIPTION OF THE INVENTION

According to the present invention, a system is described for comparing the physiological parameters of a patient which resolves the abovementioned problems.

Such system advantageously consists of a pair of detection devices, a main one and at least a secondary, which are adapted to detect the same physiological parameters in two distinct portions of the body of a patient.

In more detail, the primary detection device is adapted to be positioned at a skin portion, healthy or damaged, regarding which it is desired to monitor the perfusion state of a wound or regarding which it is desired to verify the presumed presence of circulatory disturbances.

The detected parameters will be at least those of skin temperature and, in some still more advantageous embodiments, also the pH and the skin oxygenation will be detected through dedicated sensors.

In order to avoid considering possible irregular parameters, which however are not only localized in the area to be diagnosed, but rather extended over the entire body, the present invention advantageously comprises at least a second detection device to be positioned in a healthy portion of the body of the patient, possibly far from that to be diagnosed.

Said primary detection device, entering into more detail, is constituted by a first printed circuit and by a second printed circuit arranged on a flexible support, adapted to be placed at the first skin portion to be diagnosed, healthy or damaged, of a patient, being adapted to any curve of the body. The first printed circuit is that which comprises at least a matrix of temperature sensors and possibly also pH and skin oxygenation sensors. The detected data is sent to at least a microcontroller integrated in said second printed circuit.

The secondary detection device or the secondary detection devices are configured to be equivalent to the primary one.

The microcontrollers integrated in all the detection devices send the data to a display device, wired or wireless, provided with at least a screen adapted to allow the display of the data detected by said primary detection device and by said at least a secondary detection device.

In a further preferred embodiment, the primary detection device can be advantageously enlarged, i.e. the area over which the physiological parameters are detected can be extended. By means of a connection cable arranged on each side of the perimeter of said primary detection device, an additional detection portion can be engaged with a corresponding connection cable; each additional detection portion can be aggregated at a side of the perimeter of said primary detection device. Each additional detection portion advantageously comprises a printed circuit with a matrix of sensors adapted to detect the same physiological parameters detected by the matrix of sensors comprised in the first printed circuit of the primary detection device. The printed circuit of each additional portion is advantageously configured for being connected to the printed circuit of the primary detection device by means of said connection cable, sending the detected data to the microcontroller and then to the display device.

Also the secondary detection device can be provided with the same cables of connection with the corresponding additional detection portions.

The advantages of the present invention are particularly important for post-operation use, for monitoring the healing of wounds. In addition, the detection of the aforesaid physiological parameters can be equally useful and advantageous for the diagnosis of disturbances of the venous and arterial circulation, which alter the skin temperature.

Possible further medical applications and uses of the present industrial invention patent application regard numerous medical science fields including the following, which are indicated as non-limiting examples:
plastic surgery, for carrying out the evaluation of the vitality of the skin flap in substance loss reconstruction;
traumatology, for carrying out the correct evaluation of the laceration-contusion wounds;
burns, for obtaining an improved evaluation of the deep burns based on the skin vitality;
diabetology, in the precise and circumscribed evaluation of the vital tissue, before amputation of the diabetic foot;
in surgery, in the evaluation of the vascularization at the margins of the suture, both pre- and post-operation.

The advantages offered by the present invention are evident in light of the description set forth up to now and will be even clearer due to the enclosed figures and to the relative detailed description.

### DESCRIPTION OF THE FIGURES

The invention will be described hereinbelow in at least a preferred embodiment by way of a non-limiting example with the aid of the enclosed figures, in which:

FIG. 1 schematically shows all the components of the system, object of the present invention, in the embodiment in which the communication of the data to the display device occurs in wireless form.

FIG. 2 illustrates in more detail the various electronic and sensor components of the primary detection device 10 (FIG. 2a) and of the secondary detection devices 20-20′ (FIG. 2b).

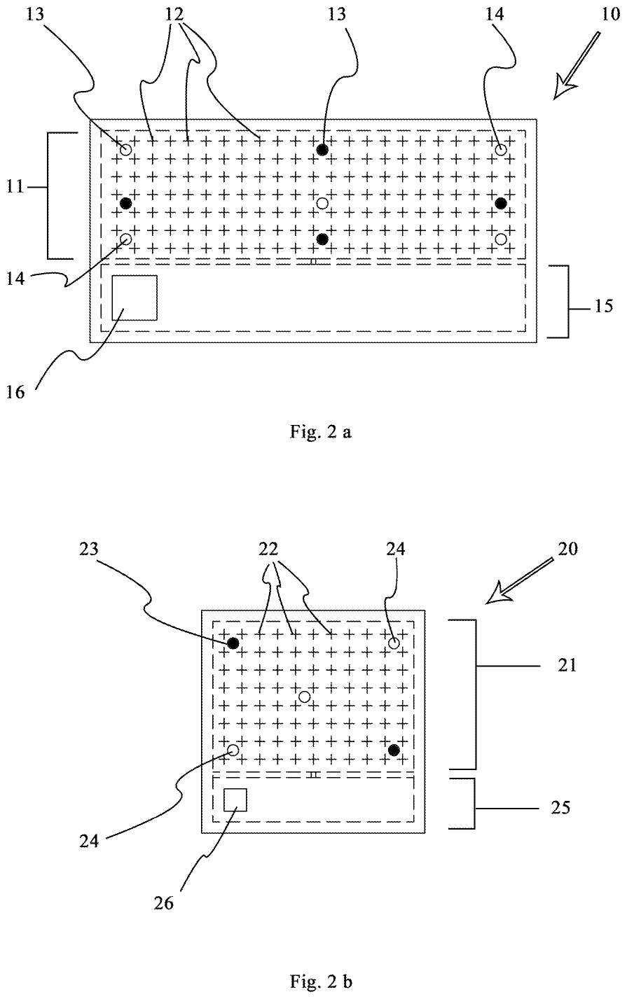

FIG. 3 shows in more detail one of the possible embodiments of the printed circuits 11-15 of the detection devices 10-20-20′.

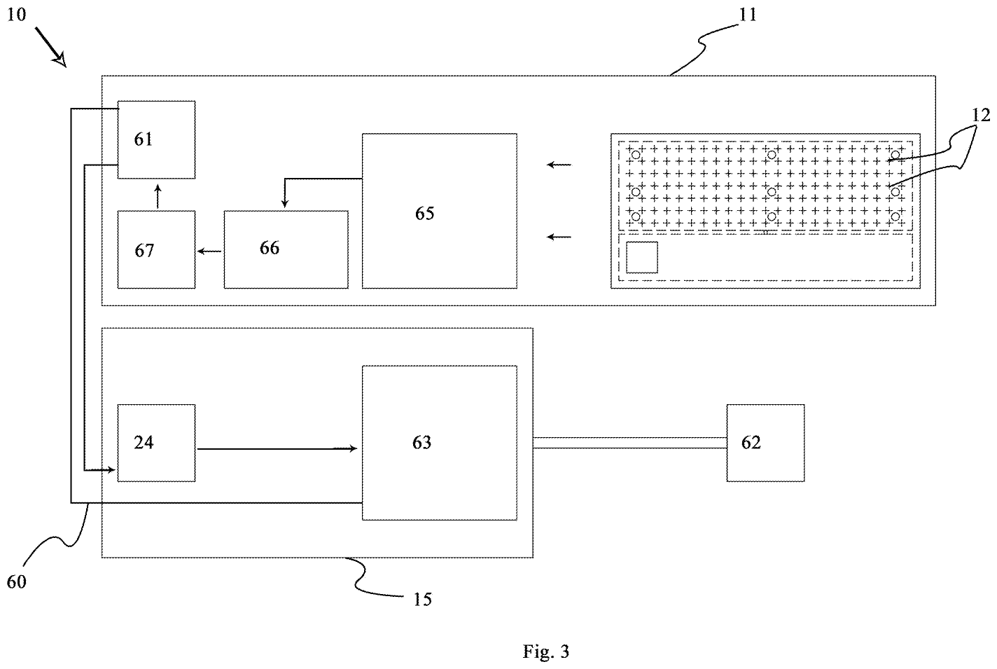

### DETAILED DESCRIPTION OF THE INVENTION

The present invention will now be illustrated as a merely exemplifying but non-limiting or non-constraining embodiment, with reference to the figures which illustrate several embodiments relative to the present inventive concept.

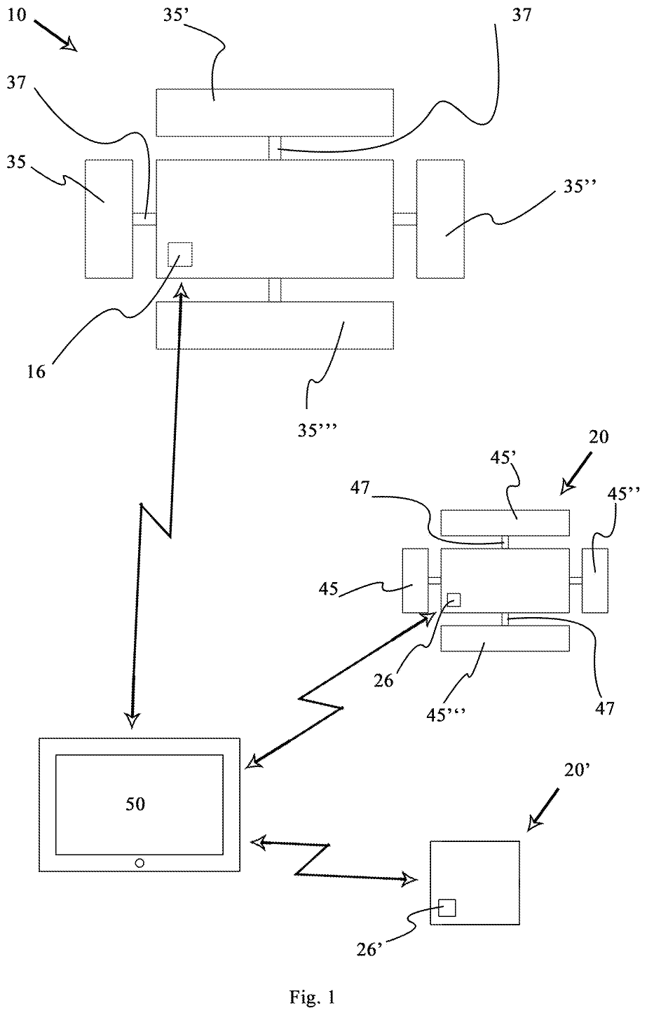

With reference to FIG. 1, the essential components of the present invention are shown, i.e.:
a primary detection device 10 constituted by a first printed circuit 11 and a second printed circuit 15 arranged on a flexible support made of polyimide so as to be adapted to any curve of the patient's body. Such primary detection device 10 is adapted to be placed at a first skin portion to be diagnosed, healthy or damaged, of a patient.
a first secondary detection device 20 constituted by a first printed circuit 21 and a second printed circuit 25 arranged on a flexible support made of polyimide so as to be adapted to any curve of the body of the patient. Such secondary detection device 20 is adapted to be placed at a second skin portion to be compared, said second portion being healthy;
a display device 50 connected in a wireless manner to the microcontrollers 16-26 26′ integrated in the detection devices 10-20-20′. The display device 50 is provided with at least a screen in order to allow the display of the data detected by the connected detection devices 10-20-20′, executing a comparison of the data detected on a healthy portion and that detected on the damaged portion or in any case on the portion to be diagnosed.

The present system can also comprise at least a second secondary detection device 20′ configured like the first secondary detection device 20 described above. From FIG. 1, one also infers the possibility to extend the area detected by each detection device 10-20-20′ by means of additional portions 35-35′-35″′-35″′; 45-45′-45″ -45″′ connected to the respective central portion by means of a connection cable 37-47 for each side of the perimeter of said detection devices 10-20-20′. Each additional detection portion 35-35′-35″-35″′; 45-45′-45″′-45″′ comprises a printed circuit with a matrix of sensors adapted to detect the same physiological parameters detected by the matrix of sensors comprised in the first printed circuit 11-21 of the central portion of said detection device 10-20-20′. The printed circuit of each additional portion 35-35′-35″-35″′; 45-45′-45″-45″′ is configured for being connected to the printed circuit of the central portion of the detection device 10-20-20′- . . . by means of said connection cable 37-47, sending the detected data to the respective microcontroller 16-26 and, finally, to the display device 50.

Entering into more detail in the technical operation of the printed circuits 11-15-21-25 of the detection devices 10-20-20′, these are preferably configured as follows.

A first printed circuit 11-21 comprises at least a matrix of temperature sensors 12-22, of acidity sensors (pH) 13-23 and of skin oxygenation sensors 14-24, as these are the physiological parameters which indicate the vitality of the skin. Said matrix comprising, preferably, 16 lines by 32 columns.

A connector 61, in which a flexible connection cable 60 is inserted, connects the first printed circuit 11-21 to the second printed circuit 15-25.

The second printed circuit 15-25 comprises:
a MCU 63 adapted to select said sensors 12-22-13-23-14-24 one at a time, sending on a bus the number of the line and of the column to be selected and adapted to receive the measurement of the sensor by reading the output of a digital analog converter 64;
selection devices 65 adapted to activate the line and the corresponding column;
a transistor in series with each sensor 12-22-13-23-14-24 adapted to select the corresponding sensor 12-22-13-23-14-24;
a NTC resistor in order to connect the power supply to a resistive divider 66;
said resistive divider 66 adapted to acquire the current from said NTC resistor and generate a voltage;
a LNA 67 adapted to scale and translate the possible output voltages from said resistive divider 66 in order to fully exploit the input dynamics of said digital analog converter 64.
USB interface means 62 adapted to transmit the digitized information to other peripheral devices.

Finally, it is clear that modifications, additions or variations that are obvious to the man skilled in the art can be made to the invention, without departing from the protective scope that is provided by the enclosed claims.

1. System for comparing the physiological parameters of a patient, adapted to acquire, process and compare detected data relative to the parameters of skin vitality in at least a pair of distinct portions of the body of a patient; said system comprising at least:
one primary detection device constituted by a first printed circuit and a second printed circuit arranged on a flexible support, adapted to be placed at a first skin portion to be diagnosed, healthy or damaged, of a patient; said first printed circuit comprising at least a matrix of temperature sensors adapted to send the detected temperature data to at least a microcontroller integrated in said second printed circuit;
at least a secondary detection device constituted by a first printed circuit and a second printed circuit arranged on a flexible support, adapted to be placed at a second skin portion, healthy, of a patient in order to detect the same data as said first skin portion; said first printed circuit comprising at least a matrix of temperature sensors adapted to send the detected temperature data to at least a microcontroller integrated in said second printed circuit;
a display device connected to said microcontrollers, wired or wireless, provided with at least a screen adapted to allow the display of the detected data by said primary detection device and by said at least a secondary detection device.

2. The system for comparing the physiological parameters of a patient, according to claim 1, further comprising a plurality of secondary detection devices adapted to detect the physiological parameters in a corresponding plurality of skin portions, sending the detected data to said display device.

3. The system for comparing the physiological parameters of a patient, according to claim 1, wherein each detection device comprises at least an acidity sensor or matrix of acidity sensors adapted to detect the corresponding data regarding the pH of the skin portion on which said detection devices are positioned, sending the corresponding data to said display device.

4. The system for comparing the physiological parameters of a patient, according to claim 1, wherein each detection device comprises at least an oxygenation sensor or matrix of oxygenation sensors adapted to detect the corresponding data regarding the skin oxygenation of the skin portion on which said detection devices are positioned, sending the corresponding data to said display device.

5. The system for comparing the physiological parameters of a patient, according to claim 1, wherein at least said primary detection device comprises at least connection cable for each side, adapted to be reversibly engaged with a corresponding cable of an additional detection portion, each of which aggregable at one side of the perimeter of said primary detection device; each additional detection portion comprising a printed circuit with a matrix of sensors adapted to detect the same physiological parameters detected by the matrix of sensors comprised in the first printed circuit of the central portion of said primary detection device; said printed circuit of each additional portion being configured for being connected to the printed circuit of the central portion of the primary detection device by means of said connection cable, sending the detected data to the microcontroller of the aforesaid central portion of the primary detection device.

6. The system for comparing the physiological parameters of a patient, according to claim 6, wherein at least a of said secondary detection devices comprises at least connection cable for each side, adapted to be reversibly engaged with a corresponding cable of an additional detection portion, each of which aggregable at one side of the perimeter of said secondary detection device; each additional detection portion comprising a printed circuit with a matrix of sensors adapted to detect the same physiological parameters detected by the matrix of sensors comprised in the first printed circuit of the central portion of said secondary detection device; said printed circuit of each additional portion being configured for being connected to the printed circuit of the central portion of the secondary detection device by means of said connection cable, sending the detected data to the microcontroller of the aforesaid central portion of the secondary detection device.

7. The system for comparing the physiological parameters of a patient, according to claim 1, wherein said first printed circuit and said second printed circuit of each detection device comprise:
a connector in which a flexible connection cable is inserted;
a plurality of sensors arranged in a matrix of at least 16 lines by 32 columns;
an MCU adapted to select said sensors one at a time, sending on a bus the number of the line and of the column to be selected and adapted to receive the measurement of the sensor by reading the output of a digital analog converter;
selection devices adapted to activate the line and the corresponding column;
a transistor in series with each sensor adapted to select the corresponding sensor;
an NTC resistor adapted to connect the power supply to a resistive divider;
said resistive divider adapted to acquire the current from said NTC resistor and generate a voltage;
an LNA adapted to scale and translate the possible output voltages from said resistive divider in order to fully exploit the input dynamics of said digital analog converter.

8. The system for comparing the physiological parameters of a patient, according to claim 1, wherein at least said primary detection device comprises USB interface means adapted to transmit the digitized data to other periphery devices.

9. The system for comparing the physiological parameters of a patient, according to claim 1, wherein said flexible supports of said detection devices are made of polyimide so as to be adapted to any curve of the patient's body.

10. A method for diagnosing the state of perfusion of a wound or, on healthy skin, for the diagnosing disturbances of the blood circulation of a patient, the method comprising providing the system of claim 1, and applying the system to perform the diagnosing.

11. The system for comparing the physiological parameters of a patient, according to claim 2, wherein each detection device comprises at least an acidity sensor or matrix of acidity sensors adapted to detect the corresponding data regarding the pH of the skin portion on which said detection devices are positioned, sending the corresponding data to said display device.

12. The system for comparing the physiological parameters of a patient, according to claim 2, wherein each detection device comprises at least an oxygenation sensor or matrix of oxygenation sensors adapted to detect the corresponding data regarding the skin oxygenation of the skin portion on which said detection devices are positioned, sending the corresponding data to said display device.

13. The system for comparing the physiological parameters of a patient, according to claim 3, wherein each detection device comprises at least an oxygenation sensor or matrix of oxygenation sensors adapted to detect the corresponding data regarding the skin oxygenation of the skin portion on which said detection devices are positioned, sending the corresponding data to said display device.

14. The system for comparing the physiological parameters of a patient, according to claim 2, wherein at least said primary detection device comprises at least connection cable for each side, adapted to be reversibly engaged with a corresponding cable of an additional detection portion, each of which aggregable at one side of the perimeter of said primary detection device; each additional detection portion comprising a printed circuit with a matrix of sensors adapted to detect the same physiological parameters detected by the matrix of sensors comprised in the first printed circuit of the central portion of said primary detection device; said printed circuit of each additional portion being configured for being connected to the printed circuit of the central portion of the primary detection device by means of said connection cable, sending the detected data to the microcontroller of the aforesaid central portion of the primary detection device.

15. The system for comparing the physiological parameters of a patient, according to claim 3, wherein at least said primary detection device comprises at least connection cable for each side, adapted to be reversibly engaged with a corresponding cable of an additional detection portion, each of which aggregable at one side of the perimeter of said primary detection device; each additional detection portion comprising a printed circuit with a matrix of sensors adapted to detect the same physiological parameters detected by the matrix of sensors comprised in the first printed circuit of the central portion of said primary detection device;
said printed circuit of each additional portion being configured for being connected to the printed circuit of the central portion of the primary detection device by means of said connection cable, sending the detected data to the microcontroller of the aforesaid central portion of the primary detection device.

16. The system for comparing the physiological parameters of a patient, according to claim 4, wherein at least said primary detection device comprises at least connection cable for each side, adapted to be reversibly engaged with a corresponding cable of an additional detection portion, each of which aggregable at one side of the perimeter of said primary detection device; each additional detection portion comprising a printed circuit with a matrix of sensors adapted to detect the same physiological parameters detected by the matrix of sensors comprised in the first printed circuit of the central portion of said primary detection device; said printed circuit of each additional portion being configured for being connected to the printed circuit of the central portion of the primary detection device by means of said connection cable, sending the detected data to the microcontroller of the aforesaid central portion of the primary detection device.

17. The system for comparing the physiological parameters of a patient, according to claim 2, wherein said first printed circuit and said second printed circuit of each detection device comprise:
a connector in which a flexible connection cable is inserted;
a plurality of sensors arranged in a matrix of at least 16 lines by 32 columns;
an MCU adapted to select said sensors one at a time, sending on a bus the number of the line and of the column to be selected and adapted to receive the measurement of the sensor by reading the output of a digital analog converter;
selection devices adapted to activate the line and the corresponding column;
a transistor in series with each sensor adapted to select the corresponding sensor;
an NTC resistor adapted to connect the power supply to a resistive divider;
said resistive divider adapted to acquire the current from said NTC resistor and generate a voltage;
an LNA adapted to scale and translate the possible output voltages from said resistive divider in order to fully exploit the input dynamics of said digital analog converter.

18. The system for comparing the physiological parameters of a patient, according to claim 3, wherein said first printed circuit and said second printed circuit of each detection device comprise:
a connector in which a flexible connection cable is inserted;
a plurality of sensors arranged in a matrix of at least 16 lines by 32 columns;
an MCU adapted to select said sensors one at a time, sending on a bus the number of the line and of the column to be selected and adapted to receive the measurement of the sensor by reading the output of a digital analog converter;
selection devices adapted to activate the line and the corresponding column;
a transistor in series with each sensor adapted to select the corresponding sensor;
an NTC resistor adapted to connect the power supply to a resistive divider;
said resistive divider adapted to acquire the current from said NTC resistor and generate a voltage;
an LNA adapted to scale and translate the possible output voltages from said resistive divider in order to fully exploit the input dynamics of said digital analog converter.

19. The system for comparing the physiological parameters of a patient, according to claim 4, wherein said first printed circuit and said second printed circuit of each detection device comprise:
a connector in which a flexible connection cable is inserted;
a plurality of sensors arranged in a matrix of at least 16 lines by 32 columns;
an MCU adapted to select said sensors one at a time, sending on a bus the number of the line and of the column to be selected and adapted to receive the measurement of the sensor by reading the output of a digital analog converter;
selection devices adapted to activate the line and the corresponding column;
a transistor in series with each sensor adapted to select the corresponding sensor;
an NTC resistor adapted to connect the power supply to a resistive divider;
said resistive divider adapted to acquire the current from said NTC resistor and generate a voltage;
an LNA adapted to scale and translate the possible output voltages from said resistive divider in order to fully exploit the input dynamics of said digital analog converter.

20. The system for comparing the physiological parameters of a patient, according to claim 5, wherein said first printed circuit and said second printed circuit of each detection device comprise:
a connector in which a flexible connection cable is inserted;
a plurality of sensors arranged in a matrix of at least 16 lines by 32 columns;
an MCU adapted to select said sensors one at a time, sending on a bus the number of the line and of the column to be selected and adapted to receive the measurement of the sensor by reading the output of a digital analog converter;
selection devices adapted to activate the line and the corresponding column;
a transistor in series with each sensor adapted to select the corresponding sensor;
an NTC resistor adapted to connect the power supply to a resistive divider;
said resistive divider adapted to acquire the current from said NTC resistor and generate a voltage;
an LNA adapted to scale and translate the possible output voltages from said resistive divider in order to fully exploit the input dynamics of said digital analog converter.
